(19) United States
(12) Patent Application Publication
(10) Pub. No.: US 2014/0213993 A1
(43) Pub. Date: Jul. 31, 2014

(54) OPHTHALMIC SURGICAL CASSETTE

(71) Applicant: Carl Zeiss Meditec AG, Jena (DE)

(72) Inventors: Christoph Kuebler, Oberkochen (DE); Martin Kraus, Huettlingen (DE)

(73) Assignee: Carl Zeiss Meditec AG, Jena (DE)

(21) Appl. No.: 14/228,968

(22) Filed: Mar. 28, 2014

(30) Foreign Application Priority Data
Sep. 28, 2011 (DE) 10 2011 114 468.8

(51) Int. Cl. A61M 1/00; A61F 9/007

(52) U.S. Cl. CPC A61M 1/0066; A61F 9/00736
USPC 604/319

(57) ABSTRACT

An ophthalmic surgical cassette acts with a peristaltic pump having a wheel defining a periphery and includes a cassette body having a first recess which, in cross section, has a circle segment shape defining a circular arc. The first recess permits the wheel of the peristaltic pump to engage therein. An aspiration line transports aspirated fluid via the pump. A second recess along the circular arc of the circle segment shape has a wall forming a fluid channel. The fluid channel has a first end connected to the aspiration line and a second end connected to a collection container supply line. An elastic cover covers the channel and is held to ensure a constant spacing of the cover to the second recess to permit fluid from the aspiration line to be transported through the channel to the container supply line via the wheel acting on the cover.

### CROSS REFERENCE TO RELATED APPLICATIONS

This application is a continuation application of international patent application PCT/EP2012/068768, filed Sep. 24, 2012, designating the United States and claiming priority from German application 10 2011 114 468.8, filed Sep. 28, 2011, and the entire content of both applications is incorporated herein by reference.

### FIELD OF THE INVENTION

The invention relates to an ophthalmic surgical cassette, and to an ophthalmic surgical system with such a cassette.

### BACKGROUND OF THE INVENTION

There are several surgical techniques for treatment of clouding of the lens, which is referred to in medicine as gray cataract. The most widely used technique is phacoemulsification, in which a thin tip is introduced into a diseased lens and is excited to vibration with ultrasound. In its immediate environment, the vibrating tip emulsifies the lens in such a way that the resulting lens fragments can be sucked through a line by a pump. When the lens has been completely emulsified, a new and artificial lens can be inserted into the empty capsular bag, such that a patient treated in this way can recover good visual acuity.

In phacoemulsification, a system is used that generally has a vibratable tip in a handpiece, a flushing line (irrigation line) for conveying irrigation fluid to the lens to be treated, and a suction line (aspiration line) for transporting emulsified lens fragments and the fluid into a collection container. The fluid and the lens fragments can be transported away via a peristaltic pump. This is a positive-displacement pump in which a pump wheel presses on a hose in which the fluid is contained. By a movement of the wheel and simultaneous pressure on the hose jacket, the fluid inside the hose is transported forward. The advantage of such a pump is that the fluid and the lens fragments are not touched directly by the pump components, and therefore the fluid cannot be contaminated by the pump. Moreover, it is also possible for small quantities to be transported relatively precisely. However, a disadvantage is that the pump wheel has to act on the hose with quite considerable force, and it is therefore necessary for the associated retention plate or cassette to be constructed so that it is torsionally rigid. When a pressure body of the pump wheel comes into engagement with the hose, abrupt pressure fluctuations or pulsations arise in the fluid inside the hose line. Since a pump wheel has several pressure bodies, several pressure pulses are introduced into the fluid on each revolution of a wheel. In continuous operation of a pump wheel, cyclical pulsations therefore arise inside the hose line. Since the pressure fluctuations of the fluid can propagate all the way to the eye, this means that, above a certain strength of the pressure fluctuations, this can lead to dangerous injuries in the eye, which absolutely have to be avoided.

United States patent application publication 2008/0114312 A1 describes a suction pump having: a pump head with a large number of projections, wherein the pump head is arranged such that is able to rotate about an axis, a ramp which is arranged near the rotating projections, wherein the ramp has a central portion, an entrance portion, and an exit portion, wherein the pump moreover has a resilient channel configured to transfer fluid when it comes into engagement with the ramp and with the large number of projections.

### SUMMARY OF THE INVENTION

It is an object to provide an ophthalmic surgical cassette which, in interaction with a peristaltic pump, allows fluid and lens fragments to be transported away with a relatively low pulsation frequency and at a low pulsation strength. Such a cassette should also be able to be made in a lightweight construction that uses a minimal amount of materials. It is a further object of the invention to provide an ophthalmic surgical system having such a cassette.

For the ophthalmic surgical cassette, the object is achieved by an ophthalmic surgical cassette configured to act with a peristaltic pump having a wheel defining a periphery, the ophthalmic surgical cassette having:
a cassette body with, on a narrow side, a first recess which is designed in cross section in the form of a circle segment having an arc, wherein the first recess is designed such that a periphery of a wheel of a peristaltic pump can engage in the first recess,
an aspiration line for transporting a fluid aspirated by means of the peristaltic pump,
wherein a second recess in the cassette body is provided along the arc of the circle segment in the radial direction, the wall of which second recess forms a fluid channel,
wherein the fluid channel is connected at a first end to the aspiration line and is connected at a second end to a collection container supply line,
wherein the cassette has a holding device for holding an elastic cover element with which the fluid channel can be covered, wherein the holding device ensures a constant distance between the cover element and the second recess, such that fluid from the aspiration line can be transported through the covered fluid channel to the collection container supply line by means of the wheel of the peristaltic pump acting on the cover element.

The cassette according to the invention has, on a narrow side, a first recess which in cross section is in the form of a circle segment having an arc. A periphery of a wheel of a peristaltic pump can engage in the first recess. A pump wheel thus acts perpendicularly on the narrow side, such that a high axial area moment of inertia of the cassette body can be exploited. This has the effect that the bending of the cassette body is only very slight. Such a construction is advantageous compared to cassettes in which the pump wheel acts on the front face of the cassette, which is arranged perpendicular to the narrow sides of the cassette. With force acting on the front face of the cassette, there is considerable bending of the cassette, and therefore, in order to reduce this effect, considerable outlay is needed to provide a torsionally rigid structure. By using the narrow side of the cassette body for the engagement of the pump wheel, the cassette body can be constructed without additional stiffening ribs or similar measures and in a simple way that cuts down on materials, and yet a high degree of torsional rigidity of the cassette is obtained during the operation of the pump and during engagement in the first recess of the cassette.

The cassette according to the invention is also provided with a second recess along the arc of the circle segment in the radial direction, the wall of which second recess forms a fluid channel, wherein the cassette has a holding device for holding an elastic cover element with which the fluid channel can be covered. It is therefore not necessary, unlike in the prior art, to transport the aspirated fluid and the lens fragments in a hose which engages with the pump wheel. Instead, the cassette has a holding device in order to hold an elastic cover element with which the fluid channel can be covered. Therefore, the pump wheel does not act on a hose placed in the cassette, but on an elastic cover element. By configuring the holding device such that it ensures a constant distance of the cover element along the second recess, the fluid to be aspirated is always transported reliably along the fluid channel by the pump wheel. As a result of the constant distance between the cover element and the second recess, the cover element does not lift away from the second recess upon engagement of a pump wheel, such that a constant cross section of the fluid channel and, therefore, a constant delivery rate of the fluid are achieved.

The constant distance between the cover element and the second recess also has the effect that, unlike in the prior art, it is not necessary to provide a pump wheel with a relatively large number of pressure bodies in order to suppress the lifting of a hose. With the cassette according to the invention, it is instead possible to achieve a constant delivery rate of the fluid using a pump wheel with only a few pressure bodies. In this way, only relatively small pressure pulses are induced in the fluid that is to be delivered, such that overall a lower pulsation frequency in the aspiration line is achieved by comparison with cassettes according to the prior art. A smaller number of pressure bodies also leads to a further advantageous effect: the fewer pressure bodies a pump wheel has, the less volume of the fluid channel is claimed by the pressure bodies, and the more fluid can be transported in a predefined length of the fluid channel. In this way, a relatively high delivery volume can be achieved even at a low speed of rotation of the pump wheel. This permits an additional reduction in the pulsation frequency, wherein a low speed of rotation of the pump wheel generally signifies a smoother delivery of fluid at a lower pulsation strength.

The holding device of the cassette preferably holds the elastic cover element via a form fit, a force fit or a cohesive bond. The holding device can use the geometry of the cover element in order to achieve a form fit. The holding device can additionally or alternatively be configured such that it holds the cover element to the cassette body as a result of a force fit, for example with an interference fit between cover element and cassette body. If the holding device and the cover element are held for example via a cohesive bond to the cassette body, the holding device can be configured for example as an adhesive.

The holding device is preferably a bead protruding from the cassette body, or a groove introduced into the cassette body, such that at least a form fit is achieved. Particularly preferably, the bead or the groove is provided on a surface perpendicular to the narrow side of the cassette body. A particularly secure form fit can be achieved by such engagement around corners. The cover element can also be connected to the cassette in one piece, for example by two-component injection molding.

According to a further embodiment, the cross section of the fluid channel varies in the longitudinal direction thereof. In the area of the fluid channel in which a pressure body of the pump wheel first comes into engagement with the cover element, the fluid channel can have a different cross section than in the adjoining area of the fluid channel. It is thus possible to considerably reduce the strength of a pulsation of the aspiration fluid.

The fluid channel at one end preferably extends onward tangentially to its normal at the end. The fluid channel can also extend onward at one end as a curved path, of which the direction of curvature is opposite to the direction of curvature of the arc of the circle segment of the first recess. The pulsation strength can likewise be reduced via a tangential or curved inlet or outlet area of the fluid channel.

According to a further embodiment of the invention, the aspiration line and/or an irrigation line provided in the cassette has a rigid wall made from the same material as the cassette body. In such a cassette, it is therefore not necessary to place a hose into the cassette for the aspiration line and/or irrigation line. Instead, the lines are formed by the cassette body. It is thus possible to reduce the time needed to make ready a cassette according to the invention for operation.

The second recess is preferably provided such that the cover element can be pressed completely into the second recess by a pressure body of the wheel of the peristaltic pump acting on the cover element. This permits reliable sealing and secure transport of the aspiration fluid without losses due to leakage.

The cassette preferably has two halves, which can be joined together to form the cassette body. The time needed for assembling such a cassette can thus be greatly reduced.

The ophthalmic surgical system according to the invention has an above-described cassette, a peristaltic pump, a control device for operating the peristaltic pump, and an input device for inputting signals for the control device.

### BRIEF DESCRIPTION OF THE DRAWINGS

The invention will now be described with reference to the drawings wherein:

FIG. 1 is an isometric view of a first embodiment of the ophthalmic surgical cassette according to the invention;

FIG. 2 is a cross-sectional view of the ophthalmic surgical cassette according to the invention;

FIG. 3 is a front view of the cassette according to the invention with a cover element attached;

FIG. 4 is a cross-sectional view of a fluid channel of the cassette according to the invention with a cover element attached with the cross-sectional view being taken along line A-A of FIG. 3;

FIGS. 5A to 5C are cross-sectional views of the fluid channel of the cassette according to the invention with a cover element attached, the latter being pressed to different extents into the fluid channel;

FIG. 6 is a schematic view of the cassette according to the invention with an attached cover element in which a pump wheel of a peristaltic pump engages;

FIG. 7 is a cross-sectional view of a second embodiment of the cassette according to the invention, with a tangential or curved inlet or outlet area of the fluid channel;

FIGS. 8A and 8B are cross-sectional views of the fluid channel of the cassette according to the invention with different geometries;

FIG. 9 is a schematic view of a cassette according to the invention, in a plan view of a peristaltic pump acting on the cassette;

FIG. 10 is a schematic view of the forces that are present on the cassette according to the invention during the engagement of a peristaltic pump according to FIG. 9;

FIG. 11 is a schematic view of the cassette according to the invention with aspiration and irrigation lines contained therein; and,

FIG. 12 is a view of the ophthalmic surgical system according to the invention.

### DESCRIPTION OF THE PREFERRED EMBODIMENTS OF THE INVENTION

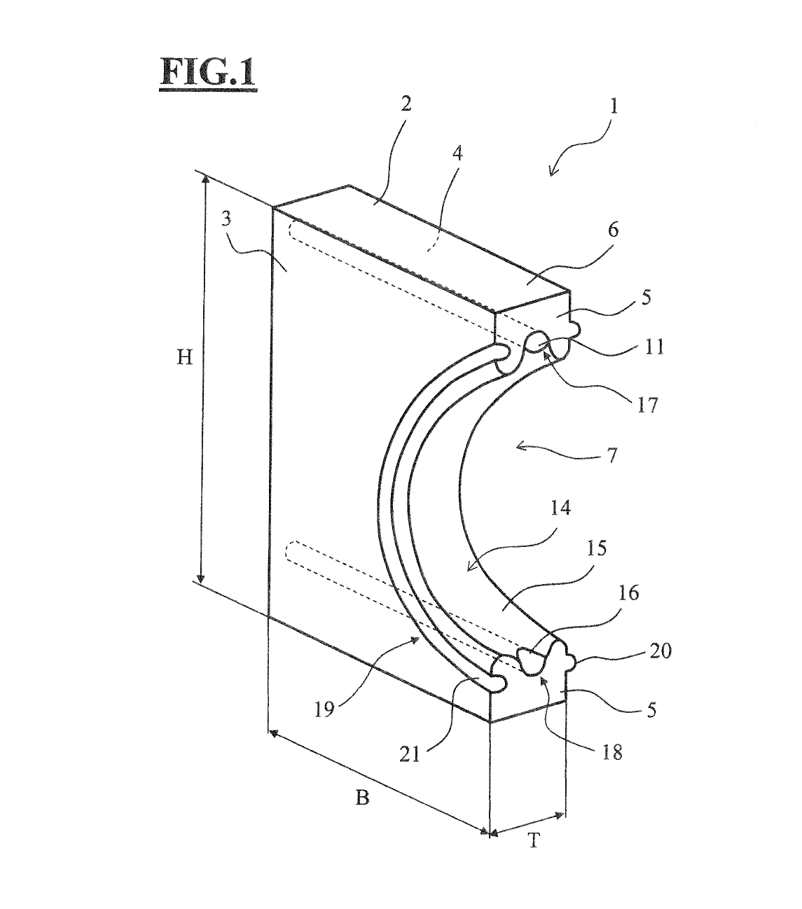

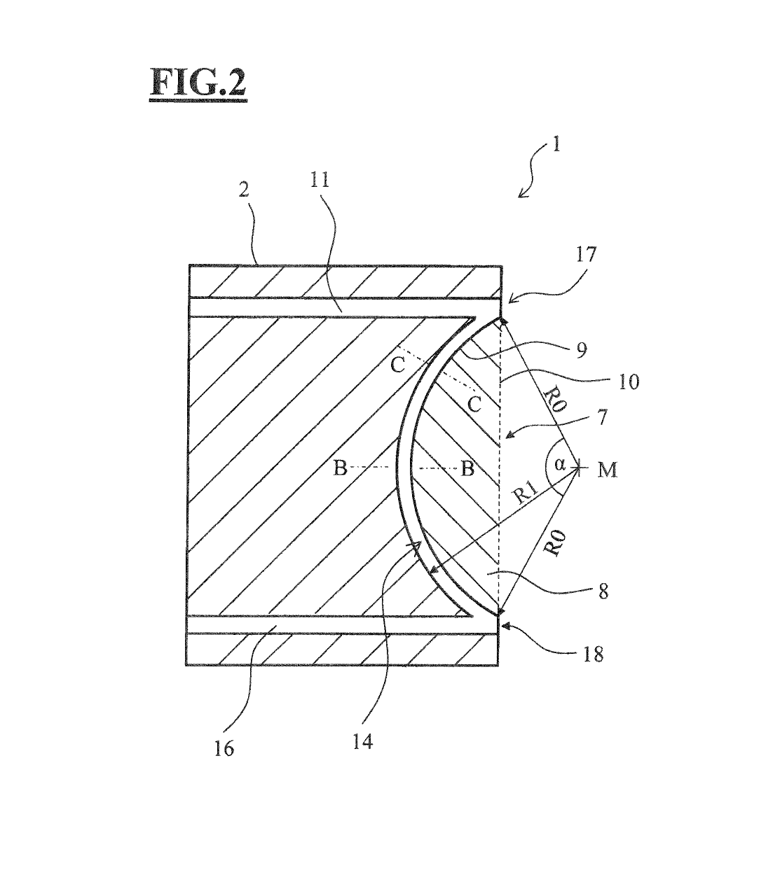

An ophthalmic surgical cassette 1 according to the invention is shown in FIG. 1 and FIG. 2. The cassette 1 has a cassette body 2, which has a front face 3 and a rear face 4. The front face 3 and rear face 4 each have a height H and width B. The cassette body 2 has a depth T, such that the rear face 4 is arranged at a distance T from the front face 3. Therefore, the substantially block-shaped cassette body 2 thus formed has a right-hand narrow side 5 and a left-hand narrow side 5, which each have a side length with the amount T and the amount H, and an upper narrow side 6 and a lower narrow side 6, which each have a side length with the amount T and the amount B.

In the cassette 1 according to the invention, a first narrow side 5 has a first recess 7 which is configured in cross section in the form of a circle segment 8 having an arc 9 (see FIG. 2).

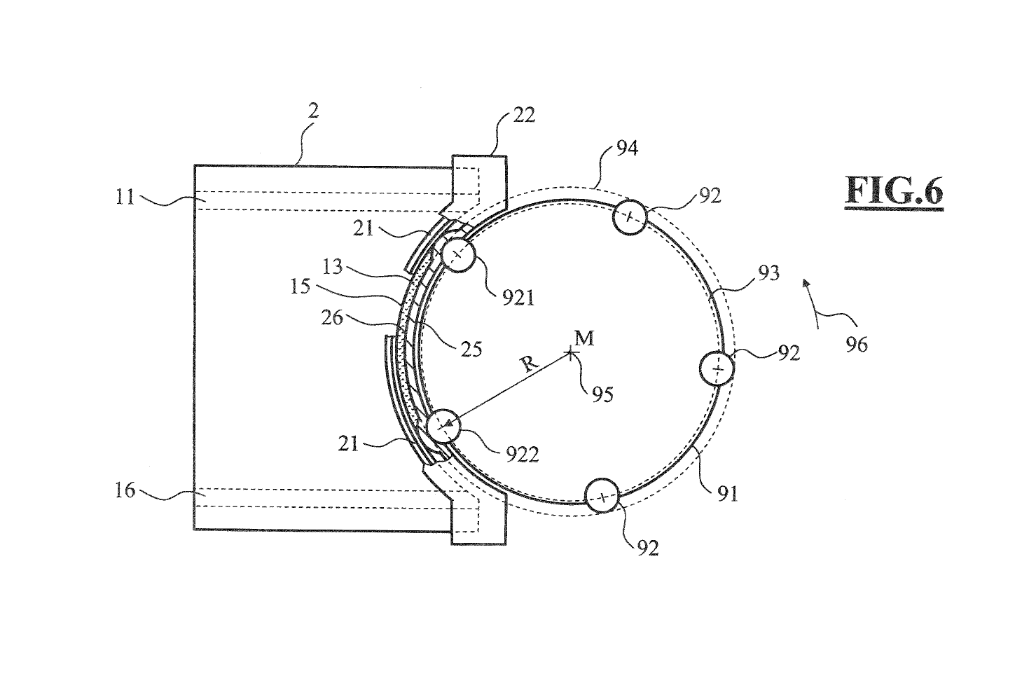

The arc 9 has a radius R0 about a circle midpoint M with a midpoint angle α. The circle segment 8 is thus defined by the arc 9 and a circle chord 10. The circle chord 10 forms a base of an equilateral triangle with legs of length R0, which intersect at the circle midpoint M. The first recess 7 has a size that is sufficient to allow a periphery of a wheel 91 of a peristaltic pump (see FIG. 6 for example) to engage in the first recess 7.

In the cassette body 2, a second recess 14 is provided along the arc 9 of the circle segment 8 in the radial direction, the wall of which second recess 14 forms a fluid channel 15. In relation to the circle midpoint M, the second recess 14 has a radius R1, which is greater than the radius RO (see FIG. 2).

The cassette body 2 also has an aspiration line 11 for transporting a fluid that is aspirated via the peristaltic pump. The aspiration line 11 is arranged in the cassette body 2 in such a way that it is connected to the fluid channel 15 at a first end 17, such that fluid from the aspiration line 11 can flow into the fluid channel 15. The cassette body 2 also has a collection container supply line 16, which is connected to the fluid channel 15 at a second end 18 of the fluid channel 15, such that aspirated fluid from the fluid channel 15 can flow through the second end 18 into the collection container supply line 16.

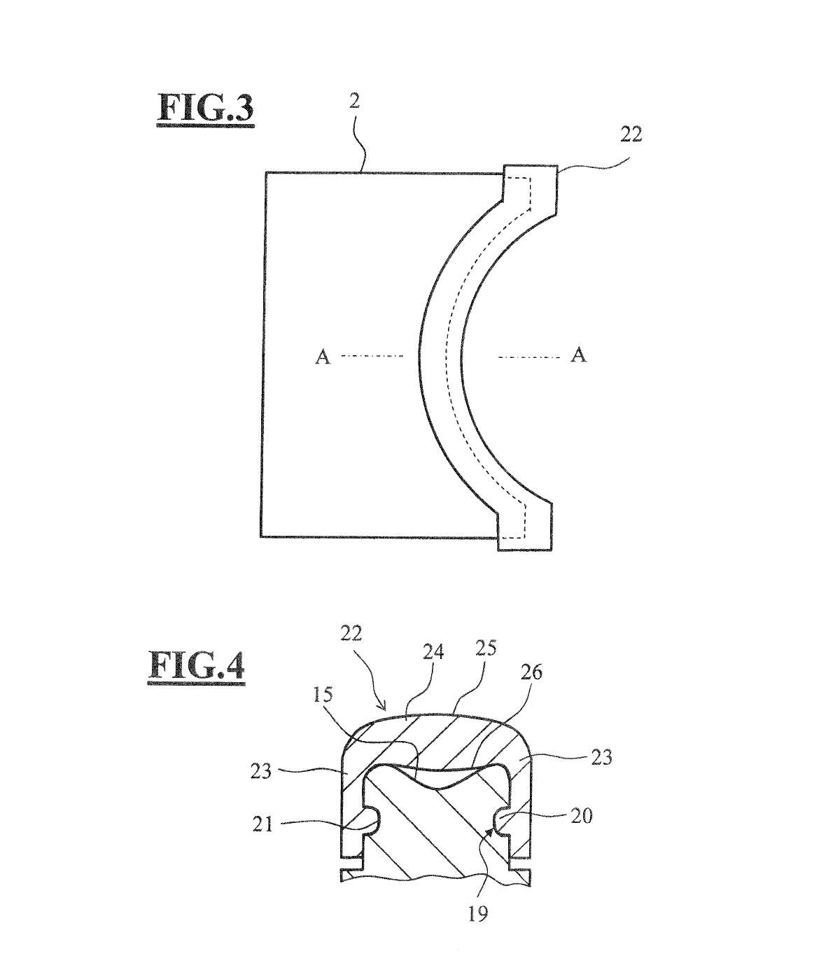

The cassette 1 also has a holding device 19 for holding an elastic cover element 22 (see FIG. 3) with which the fluid channel 15 can be covered, wherein the holding device 19 ensures a constant distance of the cover element 22 along the fluid channel 15. As can be seen from FIG. 1, the holding device can be a groove 21 introduced into the cassette body 2. However, the holding device 19 can also be a bead 20 protruding from the cassette body 2 (see FIG. 1). Moreover, the holding device 19 can also have bores introduced into the cassette body 2 or can have an adhesive film.

Generally, the holding device can hold the cover element 22 to the cassette body 2 via a form fit, a force fit or a cohesive bond. It is important here that the holding device 19 ensures a constant distance of the cover element 22 along the fluid channel 15 when a pressure body of a pump wheel of a peristaltic pump is in engagement with the elastic cover element 22.

FIG. 3 shows a front view of a cassette body 2 with an elastic cover element 22. The elastic cover element 22 is substantially arc-shaped. FIG. 4 shows a cross section, along the section line A-A in FIG. 3, of the cassette body 2 with the elastic cover element 22. The elastic cover element 22 has a substantially U-shaped cross section with two side arms 23 arranged parallel to each other, and a base 24 connecting these. The base 24 has a convex upper contour line 25 and a convex lower contour line 26. Between the lower contour line 26 and the fluid channel 15 of the cassette body 2, a free space remains in which fluid can be transported. A constant distance of the cover element 22 along the fluid channel 15 is ensured by the holding device 19, by virtue of the fact that the two side arms 23 of the cover element 22 have an outwardly protruding bead, which in each case engages in a groove 21 of the cassette body 2. A form-fit connection between the cover element 22 and the cassette body 2 is thereby achieved. The groove 21 can extend continuously and be equidistant to the fluid channel 15. However, it is also possible that the groove 21 has interruptions, such that individual groove portions are provided that are equidistant to the fluid channel 15.

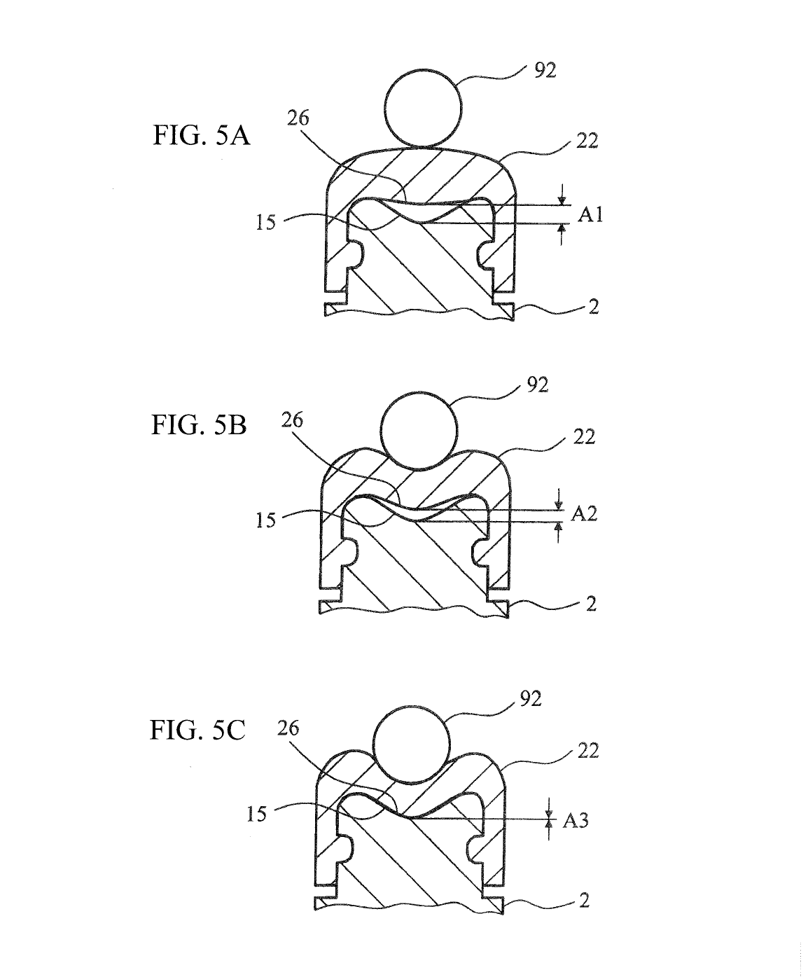

FIGS. 5A to 5C show three stages in the deformation of the elastic cover element 22 when a pressure body 92 of a wheel 91 of a peristaltic pump acts on the cover element 22 mounted on the cassette body 2. FIG. 5A shows the situation in which the pressure body 92 first comes into contact with the elastic cover element 22. Between the lower contour line 26 and the fluid channel 15 of the cassette body 2, there is still a maximum distance A1 present. FIG. 5B shows the situation when the pressure body 92 presses into the elastic cover element 22. The cover element 22 is pressed partially into the fluid channel 15, such that only a smaller distance A2 is now present between the lower contour line 26 of the cover element 22 and the fluid channel 15. FIG. 5C shows the situation in which the pressure body 92 presses the cover element 22 down so strongly that the fluid channel 15 is touched completely by the lower contour line 26 of the cover element 22. The distance between the lower contour line 26 and the fluid channel 15 is therefore A3=0. At the location where the pressure body 92 presses onto the cover element as shown in FIG. 5C, the fluid channel 15 is therefore completely sealed off.

FIG. 6 shows a front view of the cassette body 2 with an attached cover element 22, wherein pressure bodies 92 of a pump wheel 91 of a peristaltic pump engage in the cover element 22. The midpoints of the pressure bodies 92 are arranged on a circle 93 of the pump wheel 91. The pump wheel 91 has a circumferential line 94, which constitutes the outer envelope of the pressure bodies 92. The cover element 22 is dimensioned such that a pressure body 92 moving in rotation direction 96 can press the cover element 22 together in accordance with the sequence of steps shown in FIGS. 5A to 5C. A pressure body 92 thus presses the cover element 22 into the fluid channel 15 in such a way that there is no longer any distance between the cover element 22 and the fluid channel 15 (see also FIG. 5C). In FIG. 6, this situation is shown at a first pressure body 921. The same situation arises at a pressure body 922 arranged adjacent thereto. This has the effect that a fluid 13 is stored in the fluid channel 15 between the first pressure body 921 and the second pressure body 922 and, as the pump wheel 91 continues to move, this fluid 13 is transported in the drive direction 96 to the collection container supply line 16. Since the cassette body 2 in this embodiment is provided with a holding device in the form of a groove 21, which ensures that a constant distance of the cover element 22 along the fluid channel 15 is achieved, the lower contour line 26 of the cover element 22 in the area between the first pressure body 921 and the second pressure body 922 is equidistant to the deepest line of the fluid channel 15. Between the first pressure body 921 and the second pressure body 922, the cover element 22 therefore does not lift away from the fluid channel 15, which prevents the cover element 22 from extending in the form of a straight line between the two pressure bodies 921 and 922. As is shown in FIG. 6, the cover element 22 instead extends in an arc shape between the two pressure bodies 921 and 922. This has the effect that the cover element 22 between the two pressure bodies 921 and 922 is not stretched or compressed, as a result of which a very smooth transport movement and, therefore, a low pulsation strength of the aspirated fluid is achieved.

FIG. 6 shows five pressure bodies 92 of a pump wheel 91 of a peristaltic pump. Therefore, on each revolution of a pump wheel, five pressure bodies engage in the cover element 22, such that a slight pulsation or pressure fluctuation in the fluid 13 is induced five times per revolution of the pump wheel. The pulsation incidence can be reduced if the pump wheel has fewer than five pressure bodies 92, for example only three pressure bodies 92.

In previous designs in which a hose is provided instead of a cover element 22, such a small number of pressure bodies is not feasible. A hose will extend rectilinearly between the first pressure body 921 and the second pressure body 922 and does not remain in an arc-shaped contour. The hose is thus stretched and compressed, such that pronounced pressure fluctuations are introduced into the fluid. The many pressure bodies usually present then have the effect that the hose remains lying more or less in an arc shape in the cassette, such that a more or less constant delivery volume is achieved. However, the many pressure bodies also have the effect that the hose material is very greatly stretched, such that pronounced pulsations are generated in the pressure profile of the aspirated fluid.

The holding device of the cassette according to the invention not only allows the number of pressure bodies of a pump wheel to be reduced; it also permits the operation of a pump wheel with a diameter of, for example, d=200 mm, which is a much greater diameter compared to pump wheels of the prior art. Thus, a very large distance between two pressure bodies of a pump wheel can be achieved, with the result that a very large quantity of fluid can be transported along the fluid channel with only a small number of induced pressure fluctuations in the fluid.

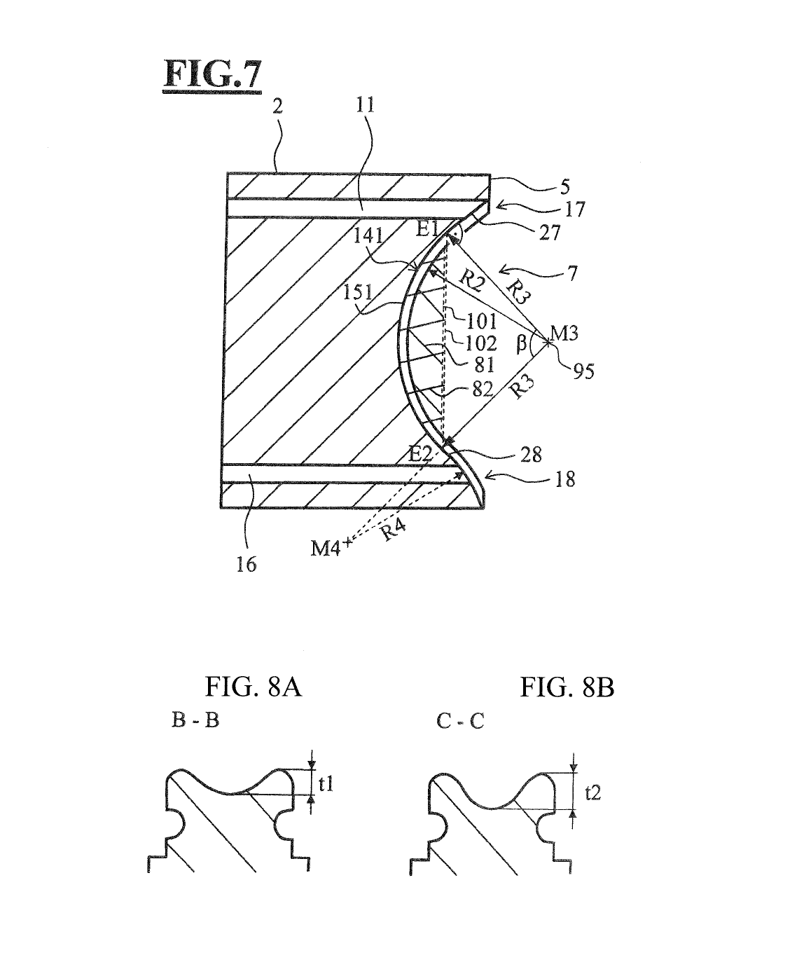

FIG. 7 shows a cross section of a further embodiment of the cassette according to the invention. In this embodiment, a circle chord 101 of a circle segment 81 with a radius R2 inside the first recess 17 does not lie in a line coinciding with the side face of a narrow side 5 but is instead offset in the direction toward the inside of the cassette body 2. Along the arc of the circle segment 81, a second recess 141 is provided in the radial direction, the wall of which second recess 141 forms a fluid channel 151. If the circle chord 101 is lengthened at both ends in its longitudinal extent, points of intersection E1 and E2 form with the lowest line of the fluid channel 151. Thus, in cross section, a circle segment 82 is obtained with an arc along the lowest line of the fluid channel 151 and with a circle chord 102 which connects the points E1 and E2 to each other, wherein the circle segment 82 has an associated midpoint angle β at a radius R3 about M3. The points E1 and E2 thus each form an end of the fluid channel 151. In this embodiment, the fluid channel 151 can now extend onward, starting from the corner E1, in such a way that, in relation to the normal at E1, it extends tangentially away from E1 (see tangent 27 in FIG. 7). The fluid channel 151 thus has an arc-shaped contour and, adjoining this, a straight contour. It is thus possible that, when a pressure body 92 traveling on a circle trajectory 93 enters the first recess 7, it will engage increasingly deeper in the elastic cover element 22 as the movement increases in movement direction 96. For the fluid in the fluid channel 151, such increasing engagement results in even lower pressure fluctuation and pulsation strength than in the first embodiment according to FIG. 2.

In a further embodiment, the fluid channel 151 extends onward at one end E2 as a curved path, of which the direction of curvature is the opposite to the direction of curvature of the arc of the circle segment 81 of the first recess 7. The fluid channel 151 thus has a first arc-shaped contour and, adjoining this, a second arc-shaped contour which, however, is curved in a direction opposite to the first arc-shaped contour. In FIG. 7, this is shown at the second end 18 of the fluid channel 151, although a curved path of this kind could also be provided in a similar way at the first end 17 of the fluid channel 151. In the embodiment shown in FIG. 7, the arc 28 has a radius R4 about a midpoint M4. In this embodiment too, it is possible that, when a pressure body 92 traveling on a circle trajectory 93 enters the first recess 7, it will engage deeper in the elastic cover element 22 as the rotation movement increases counter to the movement direction 96. For the fluid in the fluid channel 151, such increasing engagement results in a still lower pressure fluctuation and pulsation strength than in the first embodiment according to FIG. 2.

The pulsation strength of the fluid to be aspirated can also be influenced by varying the cross section of the fluid channel 15 or 151 in the longitudinal direction thereof. FIG. 8A shows a cross section of the fluid channel 15 along the line B-B of FIG. 2. The fluid channel 15 has a concave contour with a depth t1. FIG. 8B shows a cross section of the fluid channel 15 along the section line C-C of FIG. 2, wherein the fluid channel has a concave contour with a greater depth t2.

This variation of the cross section of the fluid channel in the longitudinal direction thereof is just one example, and any other geometries are possible, for example a triangular contour or a trapezoid contour.

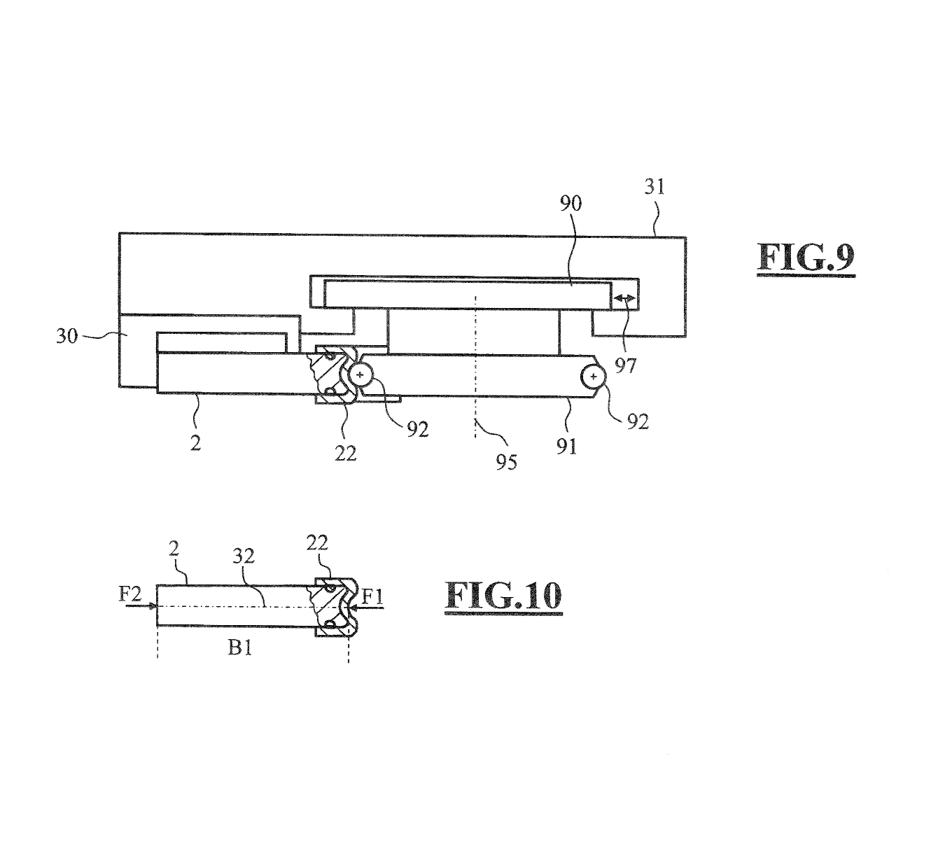

FIG. 9 shows a plan view of the cassette 1 according to the invention, wherein a pump wheel 91 of a peristaltic pump 90 acts on a cover element 22 mounted on the cassette body 2. The cassette 1 is located in a cassette seat 30, which is connected to a console 31. The pressure bodies 92 of the pump wheel 91 rotate about a drive axis 95 of the peristaltic pump 90, wherein the peristaltic pump 90 is moveable sideways (see double arrow 97). A contact between one of the pressure bodies 92 and the cover element 22 thus takes place in such a way that, after the cassette has been inserted into the cassette seat 30, the peristaltic pump 90 is moved sideways toward the cover element. Correspondingly, the cassette is exchanged by first of all moving the peristaltic pump 90 toward the right away from the cassette until the cover bodies 92 and the cover element 22 no longer touch.

FIG. 10 shows a cassette 1 analogous to FIG. 9 and indicates force vectors F1 and F2 acting on the cassette 1. When a pressure body 92 of the wheel 91 of the peristaltic pump 90 acts on the cover element 22, the force vector F1 lies in a line with the center plane 32 of the cassette 1. The cassette seat 30 counters the acting force F1 with a reaction force F2, which likewise lies in a line with the center axis 32 of the cassette. The forces F1 and F2 act at a distance B1 from each other. With such force distribution, the axial area moment of inertia of the cassette body is relatively high, since the distance B1 in the calculation of the axial area moment of inertia is in the third power. The cassette body 2 can therefore be of a relatively simple structure, and no great outlay is needed for the construction of stiffening ribs or the like.

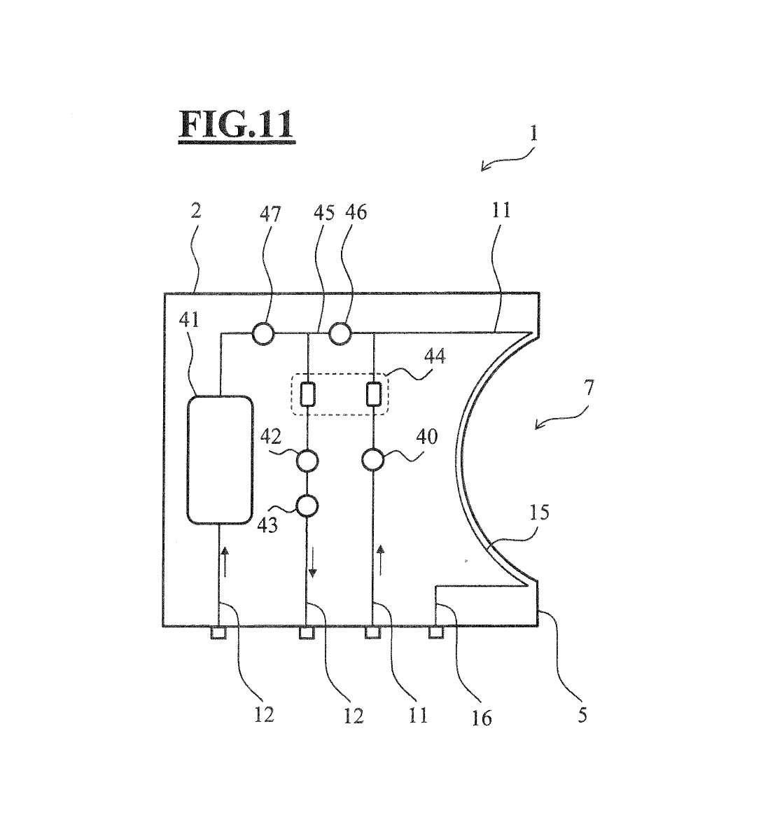

FIG. 11 shows a schematic view of the cassette 1 according to the invention. The first recess 7 is provided on the first narrow side 5, and several lines run inside the cassette body 2. The cassette body 2 has an aspiration line 11 and a switching valve 40, with which a through-flow of fluid can be reliably interrupted or enabled. The aspiration line 11 is connected to the fluid channel 15, which is adjoined by the collection container supply line 16. In addition, the cassette body 2 has an irrigation line 12, with which an irrigation fluid container 41 can be filled. The irrigation line 12 is then routed to an outlet of the cassette body 2, wherein a pressure meter 42 for measuring a pressure in the irrigation line 12 and a volumetric flow meter 43 for measuring the volumetric flow in the irrigation line 12 are provided. In addition, a differential pressure meter 44 is provided, with which a differential pressure between the aspiration line 11 and the irrigation line 12 can be measured. The irrigation line 12 is connected to the aspiration line 11 via a connection line 45 and a reflux valve 46. The aspiration line 11 can thus be filled by the supply of irrigation fluid when the reflux valve 46 is opened.

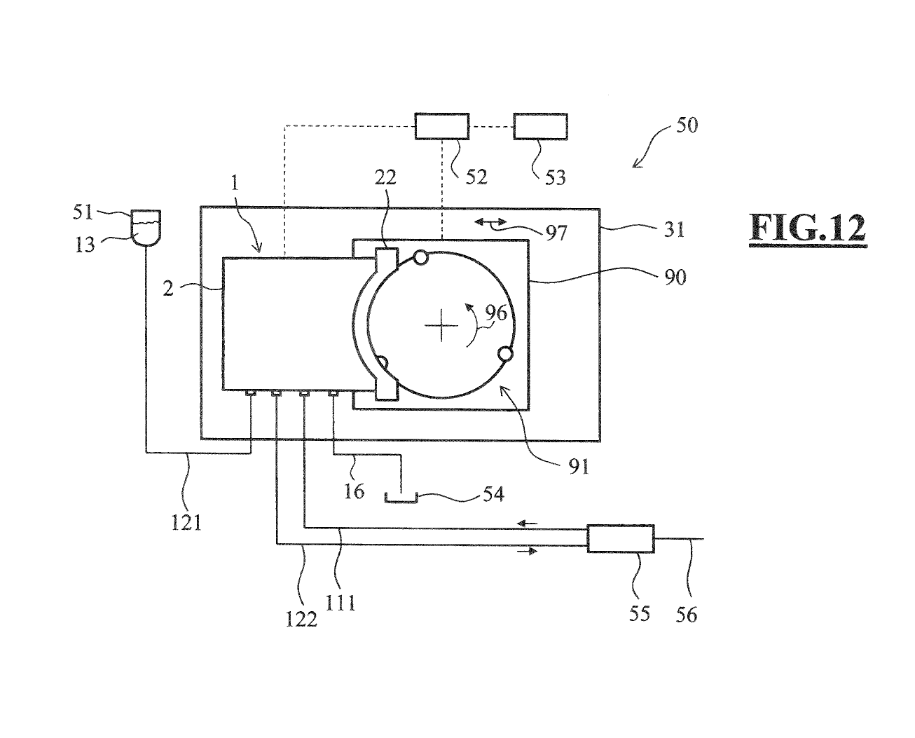

FIG. 12 shows an ophthalmic surgical system 50, which has a cassette 1, a console 31, and a peristaltic pump 90 with a pump wheel 91. The pump wheel 91 engages in the first recess 7 of the cassette body 2. A movement of the peristaltic pump 90 in the direction toward the cassette body 2 or away from this cassette body 2 according to the double arrow 97 can take place via a control device 52, which is coupled to an input device 53. The control device 52 is also suitable for adjusting the direction of rotation of the pump wheel 91, the duration of a rotation of the pump wheel 91, and a rotation speed of the pump wheel 91. An irrigation fluid container 51 holds an irrigation fluid 13, which can pass through a first supply line 121 to the irrigation line 12 in the cassette body 2. The irrigation fluid leaves the irrigation line 12 of the cassette body 2 to pass into a second supply line 122, which is connected to a phaco handpiece 55. Fluid to be aspirated passes through a third supply line 111 to the aspiration line 11 in the cassette body 2. During a movement of the pump wheel 91 in drive direction 96, the aspiration fluid is conveyed through the fluid channel 15 to the collection container supply line 16, until it emerges from the cassette body 2 and is passed through the collection container supply line 16 to a collection container 54. The control device 52 is also suitable for controlling the switching valves 40, 46 and 47 and for recording and processing the measured values of the pressure meter 42, of the volumetric flow meter 43 and of the differential pressure meter 44.

It is understood that the foregoing description is that of the preferred embodiments of the invention and that various changes and modifications may be made thereto without departing from the spirit and scope of the invention as defined in the appended claims.

What is claimed is:

1. An ophthalmic surgical cassette configured to act with a peristaltic pump having a wheel defining a periphery, the ophthalmic surgical cassette comprising:
a cassette body having a narrow side defining a first recess which, in cross section, has a circle segment shape defining a circular arc;
said first recess being configured so as to permit the periphery of the wheel of the peristaltic pump to engage therein;
an aspiration line configured to transport a fluid aspirated via the peristaltic pump;
said cassette body defining a second recess along said circular arc of said circle segment shape;
said second recess having a wall forming a fluid channel in radial direction;
a collection container supply line;
said fluid channel having a first end connected to said aspiration line and a second end connected to said collection container supply line;
an elastic cover configured to cover said fluid channel; and,
a holding arrangement for holding said elastic cover to ensure a constant distance of said cover to said second recess so as to permit fluid from said aspiration line to be transported through said covered fluid channel to said collection container supply line via the wheel of the peristaltic pump acting on said cover.

2. The ophthalmic surgical cassette of claim 1, wherein said holding arrangement holds said elastic cover on said cassette body via one of a form-fit connection, a frictional connection, and a material-to-material connection.

3. The ophthalmic surgical cassette of claim 1, wherein said holding arrangement is one of a bead protruding from said cassette body and a groove formed in said cassette body.

4. The ophthalmic surgical cassette of claim 3, wherein said cassette body defines a surface perpendicular to said narrow side; and, said one of said bead and said groove are provided on said surface.

5. The ophthalmic surgical cassette of claim 1, wherein said fluid channel defines a longitudinal direction and a cross section which varies in said longitudinal direction.

6. The ophthalmic surgical cassette of claim 1, wherein:
said fluid channel defines a normal at said first end;
said fluid channel extends tangentially referred to said normal at said first end.

7. The ophthalmic surgical cassette of claim 1, wherein:
said fluid channel defines a normal at said first end;
said fluid channel extends with a curved course at said first end;
said arc of said first recess has a first curvature direction; and,
said curved course has a second curvature direction opposite to said first curvature direction.

8. The ophthalmic surgical cassette of claim 1, wherein said aspiration line has a rigid wall made the same material as said cassette body.

9. The ophthalmic surgical cassette of claim 1 further comprising:
an irrigation line disposed in said cassette body; and,
at least one of said irrigation line and said aspiration line has a rigid wall made of the same material as said cassette body.

10. The ophthalmic surgical cassette of claim 1, wherein the wheel of the peristaltic pump has a pressure body; and, said cover is configured to be acted by said pressure body so as to be pressed completely into said second recess.

11. The ophthalmic surgical cassette of claim 1, wherein said cassette body includes two halves configured to be joined to form said cassette body.

12. An ophthalmic surgical system comprising:
a peristaltic pump;
a control unit for operating said peristaltic pump;
an input device configured for inputting signals for said control unit;
an ophthalmic surgical cassette including a cassette body having a narrow side defining a first recess which, in cross section, has a circle segment shape defining a circular arc;
said first recess being configured so as to permit the periphery of the wheel of the peristaltic pump to engage therein;
said ophthalmic surgical cassette further including an aspiration line configured to transport a fluid aspirated via the peristaltic pump;
said cassette body defining a second recess along said circular arc of said circle segment shape;
said second recess having a wall forming a fluid channel in radial direction;
said ophthalmic surgical cassette further including a collection container supply line, an elastic cover configured to cover said fluid channel, and a holding arrangement for holding said elastic cover;
said fluid channel having a first end connected to said aspiration line and a second end connected to said collection container supply line; and,
said holding arrangement being configured to ensure a constant distance of said cover to said second recess so that fluid is transportable via the wheel of the peristaltic pump acting on said cover from said aspiration line through said covered fluid channel to said collection container supply line.
